SEQUENCE LISTING (1 sequence)

SEQ ID NO: 1 - protein, 11 aa, Artificial Sequence
  G PROTEIN COUPLED RECEPTOR 4, HUMAN
ETPLTSKRNST

(19) United States
(12) Patent Application Publication
(10) Pub. No.: US 2006/0067931 A1
(43) Pub. Date: Mar. 30, 2006

(54) Receptor for lysophosphatidylcholine in vascular endothelial cells and use thereof

(76) Inventor: Hazel Lum, Chicago, IL (US)

Correspondence Address: WELSH & KATZ, LTD, 120 S RIVERSIDE PLAZA, 22ND FLOOR, CHICAGO, IL, 60606, US

(21) Appl. No.: 11/233,122

(22) Filed: Sep. 22, 2005

Related U.S. Application Data
(60) Provisional application No. 60/612,991, filed on Sep. 25, 2004.

(51) Int. Cl. A61K 39/395; C07K 16/28

(52) U.S. Cl. 424/133.1; 530/388.22

(57) ABSTRACT

A paratope-containing molecule that specifically binds to human GPR4 is disclosed. That molecule preferably specifically binds to an epitope present in the C-terminal 40 residues of human GPR4. Methods of using the paratope-containing molecules and a kit containing the same are also disclosed.

### CROSS-REFERENCE TO RELATED APPLICATION

This application claims benefit of U.S. application Ser. No. 60/612,991 filed Sep. 25, 2004.

### STATEMENT REGARDING FEDERALLY SPONSORED RESEARCH OR DEVELOPMENT

The present invention was made in part with U.S. Government support under grant number HL62649 from the National Institutes of Health, National Heart, Lung and Blood Institute. The U.S. Government has certain rights to this invention.

### TECHNICAL FIELD

The present invention contemplates a method for modulating the signaling of G protein-coupled receptors, and influencing diverse physiological processes including cell proliferation, autoimmunity and inflammation. More particularly, the present invention relates to the identification of the G protein-coupled receptors (GPCR) in microvascular endothelial cells that respond to inflammatory stress by lysophosphatidylcholine (LPC) and methods for detecting such LPC-specific GPCR, methods for inhibiting the inflammatory response of the LPC-bound GPCR. The invention also contemplates an antibody to GPR4, a GPCR that is responsive to LPC in endothelial cells and the use of that antibody.

### BACKGROUND OF THE INVENTION

Bioactive lysophospholipids regulate a wide variety of cellular activities including proliferation, smooth muscle contraction, wound healing, tumor cell invasiveness and inflammation. LPC, a combination of lysophosphatidyl acid and choline is an important example of a bioactive lysophospholipid. Formation of lysophospholipids is enhanced during oxidation of low density lipoprotein (LDL) and under inflammatory conditions.

Several inflammation-related diseases such as endometriosis (Murphy, A A, et al, J Clin Endocrinol Metab, 83:2110-2113, 1998), ovarian cancer (Okita, M D C, et al, Int J Cancer, 71:31-34, 1997), asthma and rhinitis (Mehta, D et al, Am Rev Respir Dis, 142:157-161, 1990) are associated with elevated levels of LPC. LPC plays a role in atherosclerosis and is implicated in the pathogenesis of the autoimmune disease Systemic Lupus Erythematosus (SLE) (Lusis, A J, Nature, 407:233-241, 2000; Wu, R, et al, Lupus, 8:142-150, 1999; Wu, R, et al, Clin Exp Immunol, 115:561-566, 1999). Both diseases can be regarded as chronic inflammatory conditions. Oxidatively modified phospholipids are increasingly recognized as autoantigens instrumental in their initiation and progression (Romero, F I, et al, Lupus, 9:206-209, 2000; Iuliano, L, et al, Blood, 90:3931-3935, 1997; Koh, J S, et al, J Immunol, 165:4190-4201, 2000).

LPC is produced by the action of phospholipase A₂ on phosphatidylcholine that promotes inflammatory effects including upregulation of endothelial cell adhesion molecules and growth factors, chemotaxis of monocytes, and stimulation of macrophage activation (Kume, N, et al, J Clin Invest, 90:1138-1144, 1992; Kume, N, et al, J Clin Invest, 93:907-911, 1994; Quinn, M T, et al, Pro Natl Acad Sci, USA, 84:2995-2998, 1994; Yamamoto, N, et al, J Immunol, 147:273-280, 1991). Although its mechanism of action is poorly understood, LPC exerts both stimulatory and inhibitory effects upon several intracellular signaling molecules, supporting a role for LPC as an intracellular second messenger (Prokazova, N V, et al, Biochemistry (Moscow), 63:31-37, 1998; Nishizuka, Y, Science, 258:607-614, 1992; Flavahan, N A, Am J Physiol, 264:H722-H727, 1993; Okajima, F, et al, Biochem J, 336:491-500, 1998).

Unlike other lysophospholipids, it was thought that LPC actions were not mediated through specific cellular receptors such as membrane-bound GPCRs, a view that arose from the cell lytic properties of extracellular LPC and its abundance in cell membranes and body fluids (Lee, M J, et al, Science, 279:1552-1555, 1998; Okajima, F, et al, Biochemical Journal, 336:491-500, 1998; Okita, M, et al, Int J Cancer, 71:31-34, 1997). Although studies have demonstrated G protein-dependent cellular responses to LPC, no specific high affinity LPC receptor has yet been identified (Yuan, Y, et al, J Biol Chem, 271:27090-27098, 1996; Okajima, F, et al, Biochemical Journal, 336:491-500, 1998).

Interest in both LPC and the GPCR family of receptors continues to increase due to data that suggest that they may be targets for new diagnostic and therapeutic modalities. For example, there is considerable focus on GPCRs expressed in the hematopoietic and lymphoid systems as many have been shown to play pivotal roles in the regulation of hematopoiesis and immune function. Receptor/ligand relationships within the GPCR family exhibit significant promiscuity, with many receptors recognizing more than one ligand and vice versa. This is especially true among chemokine receptors.

Consequently, there is a need in the art for the identification of both the receptors for LPC and related molecules as well as the ligands for orphan GPCRs such as GPR4 in the vascular endothelium. With such identification of the LPC mechanism in vascular endothelium, agonists and antagonists can be identified to serve as therapies for inflammatory diseases.

Definitions

As used herein, the term “anti-GPR4 antibody” is defined as an antibody that is capable of binding to GPR4.

“Polymerase chain reaction” or “PCR” refers to a procedure or technique in which minute amounts of a specific piece of nucleic acid, RNA and/or DNA, are amplified as described in U.S. Pat. No. 4,683,195 issued Jul. 28, 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol. 51:263 (1987); Erlich, ed., PCR Technology (Stockton Press, NY, 1989). As used herein, PCR is considered to be one, but not the only, example of a nucleic acid polymerase reaction method for amplifying a nucleic acid test sample comprising the use of a known nucleic acid as a primer and a nucleic acid polymerase to amplify or generate a specific piece of nucleic acid.

“Antibodies” (Abs) and “immunoglobulins” (Igs) are glycoproteins having the same structural characteristics. Although antibodies exhibit binding specificity to a specific antigen, immunoglobulins include both antibodies and other antibody-like molecules that lack antigen specificity. Polypeptides of the latter kind are, for example, produced at low levels by the lymph system and at increased levels by myelomas.

“Native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond, whereas the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Clothia et al., J. Mol. Biol. 186:651 (1985); Novotny and Haber, Proc. Natl. Acad. Sci., USA 82:4592 (1985)).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. Variability is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a beta-sheet configuration, connected by three CDRs, which form loops connecting, and in some cases forming part of the beta-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

Papain digestion of antibodies produces two identical antigen-binding fragments, called “Fab” fragments, each with a single antigen-binding site (paratope), and a residual “Fc” fragment, whose name reflects its ability to crystallize readily. Pepsin treatment yields an F(ab′)₂ fragment that has two antigen-combining sites and is still capable of cross-linking antigens.

“Fv” is the minimum antibody fragment that contains a complete antigen-recognition and -binding site. In a two-chain Fv species, this region comprises a dimer of one heavy- and one light-chain variable domain in tight, non-covalent association. In a single-chain Fv species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv see Pluckthun, in The Pharmacology of Monoclonal Antibodies, vol. 113, Rosenburg and Moore eds., Springer-Verlag, New York, pp. 269-315 (1994).

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′).sub.2 antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG₁, IgG₂, IgG₃, IgG₄, IgA₁, and IgA₂. The heavy-chain constant domains that correspond to the different classes of immunoglobulins are called alpha, delta, epsilon, gamma, and mu, respectively. The subunit structures and three-dimensional configurations of different classes of immunoglobulins are well known.

The term “antibody” specifically covers monoclonal and polyclonal antibodies, including antibody fragment clones.

“Antibody fragments” comprise a portion of an intact antibody, generally the antigen binding or variable region (paratope) of the intact antibody. Examples of antibody fragments include Fab, Fab′, F(ab′)₂, and Fv fragments; diabodies; single-chain antibody molecules, including single-chain Fv (scFv) molecules; and multispecific antibodies formed from antibody fragments.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc Natl Acad Sci, USA, 90:6444-6448 (1993).

The term “monoclonal antibody” as used herein refers to an antibody (or antibody fragment) obtained from a population of substantially homogeneous antibodies; i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or can be made by recombinant DNA methods (see, e.g., U.S. Pat. No. 4,816,567). The “monoclonal antibodies” also include clones of antigen-recognition and binding-site containing antibody fragments (Fv clones) isolated from phage antibody libraries using the techniques described in Clackson et al., Nature, 352:624-628 (1991) and Marks et al., J Mol Biol, 222:581-597 (1991), for example.

The antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, whereas the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567 to Cabilly et al.; Morrison et al., Proc. Natl. Acad. Sci. USA, 81:6851-6855 (1984)).

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In general, a humanized antibody comprises substantially all of at least one, and typically two, variable domains, in which all or substantially all of the CDR regions correspond to those of a non-human immunoglobulin and all or substantially all of the FR regions are those of a human immunoglobulin sequence. A humanized antibody optimally also comprises at least a portion of an immunoglobulin constant region (Fc), typically that of a human immunoglobulin. For further details, see Jones et al., Nature, 321:522-525 (1986); Reichmann et al., Nature, 332:323-329 (1988); and Presta, Curr Op Struct Biol, 2:593-596 (1992). The humanized antibody includes a Primatized™ antibody wherein the antigen-binding region of the antibody is derived from an antibody produced by immunizing macaque monkeys with the antigen of interest.

The term “humanized immunoglobulin” as used herein similarly refers to an immunoglobulin comprising portions of immunoglobulins of different origin, wherein at least one portion is of human origin.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv see Pluckthun, in The Pharmacology of Monoclonal Antibodies, vol. 113, Rosenburg and Moore eds., Springer-Verlag, New York, pp. 269-315 (1994).

An “isolated” antibody is one that has been identified and separated and/or recovered from a component of its natural environment. Contaminant components of its natural environment are materials that can interfere with diagnostic or therapeutic uses for the antibody, and may include enzymes, hormones, and other proteinaceous or nonproteinaceous solutes. In preferred embodiments, the antibody is purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated antibody molecules include the antibody in situ within recombinant cells because at least one component of the antibody's natural environment is not present. Ordinarily, however, isolated antibody is prepared by at least one purification step.

“Treatment” refers to both therapeutic treatment and prophylactic or preventative measures. Those in need of treatment include those already with the disorder as well as those in which the disorder is to be prevented.

“Mammal” for purposes of treatment refers to any animal classified as a mammal, including humans, domestic and farm animals, and zoo, sports, or pet animals, such as dogs, horses, cats, cows, and the like. Preferably, the mammal is human.

As used herein, the terms “each member of the group consisting of” and “each of” are synonymous.

As used herein, the terms “any member of the group consisting of” and “any of” are synonymous.

### BRIEF SUMMARY OF THE INVENTION

The present invention relates to an antibody (immunoglobulin) or functional fragment thereof (e.g., an antigen-binding fragment or paratope-containing molecule) that binds to a mammalian G-protein coupled receptor, (also referred to as GPR4) or portion of the receptor (anti-GPR4). In one embodiment, the antibody of the present invention or fragment thereof has specificity for human GPR4 or a portion thereof. In another embodiment, the antibody or fragment of the invention inhibits (reduces or prevents) binding of a ligand (e.g., LPC and other related compounds such as lysophospholipid, sphingosylphosphorylcholine (Xu, Y. Biochim Biophys Acta 1582:81-88, 2002)) to the receptor and inhibits one or more functions associated with binding of the ligand to the receptor (e.g., leukocyte trafficking). For example, as described herein, antibodies and fragments thereof of the present invention that bind human GPR4 or a portion thereof can block binding of LPC to the receptor and inhibit function associated with binding of the LPC to the receptor.

In a preferred embodiment, an antibody of the invention or fragment thereof has the same or similar epitopic specificity as the polyclonal antibody (pAb). Functional fragments of the foregoing antibodies are also contemplated. Production of monoclonal antibodies to similar receptor fragments is envisioned.

The present invention also relates to an antibody or functional fragment thereof (e.g., an antigen-binding fragment or a paratope-containing molecule) that binds to a mammalian GPR4 or portion of the receptor and provides increased fluorescent staining intensity of GPR4 or compositions comprising GPR4 relative to other anti-GPR4 antibodies. In one embodiment, the antibody is a polyclonal antibody or a monoclonal antibody that can compete with a ligand such as lysophosphatidylcholine (LPC) that binds to human GPR4 or a portion of human GPR4.

The present invention also relates to a humanized immunoglobulin or humanized antibody that binds mammalian GPR4 (e.g., human GPR4, murine GPR4). The immunoglobulin comprises an antigen-binding region of nonhuman origin (e.g., rodent) and at least a portion of an immunoglobulin of human origin (e.g., a human framework region, a human constant region or portion thereof). For example, the humanized antibody can comprise portions derived from an immunoglobulin of nonhuman origin with the requisite specificity, such as a mouse, and from immunoglobulin sequences of human origin (e.g., a chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized immunoglobulin of the present invention is an immunoglobulin containing one or more immunoglobulin chains comprising a CDR of nonhuman origin (e.g., one or more CDRs derived from an antibody of nonhuman origin) and a framework region derived from a light and/or heavy chain of human origin (e.g., CDR-grafted antibodies with or without framework changes).

The present invention further contemplates a method of inhibiting the interaction of a cell bearing mammalian (e.g., human, non-human primate or murine) GPR4 with a ligand thereof, comprising contacting the cell with an effective amount of an antibody or functional fragment thereof that binds to a mammalian GPR4 or a portion of GPR4. Suitable cells include vascular endothelial cells and other cells expressing GPR4, such as a recombinant cell expressing GPR4 or portion thereof (e.g., transfected cells).

Another embodiment of the invention relates to a method of inhibiting the interaction of a cell bearing mammalian GPR4 with a LPC, comprising contacting the cell with an effective amount of an antibody or functional fragment thereof that binds to GPR4 or a portion of that receptor. Furthermore, the invention relates to a method of inhibiting a function associated with binding of LPC to GPR4, comprising administering an effective amount of an antibody or functional fragment thereof that binds to mammalian GPR4 or a portion of said receptor.

Another aspect of the invention is a method of identifying expression of a mammalian GPR4 or portion of the receptor by a cell present in a body sample such as a tissue sample. According to the method, a composition comprising a body sample (e.g., a cell or fraction thereof such as a membrane fraction) is contacted with paratope-containing molecules of the invention such as an antibody or functional fragment thereof (e.g., 2D4) that binds to a mammalian GPR4 protein or portion of the receptor under conditions appropriate for binding of the antibody thereto. The contact is maintained for a time period sufficient for the paratope-containing molecules and GPR4 present to immunoreact to form a complex (immunoreaction product or immunocomplex), and the formation of a complex between the paratope-containing molecules and the protein or portion thereof is detected. Detection of the complex, directly or indirectly, indicates the presence of the receptor or portion thereof on the cell or fraction thereof.

The present invention also relates to a kit for use in detecting the presence of GPR4 or a portion thereof in a biological sample, comprising paratope-containing molecules that bind to a mammalian GPR4 or a portion of said receptor, and one or more ancillary reagents (an indicating means) suitable for detecting the presence of an immunocomplex formed between GPR4 and the paratope-containing molecules.

Also encompassed by the present invention are methods of identifying additional ligands or other substances that bind a mammalian GPR4 protein, including inhibitors and/or promoters of mammalian GPR4 function. For example, agents having the same or a similar binding specificity as that of an antibody of the present invention or functional fragment thereof can be identified by a competition assay with said antibody or fragment. Thus, the present invention also encompasses methods of identifying ligands or other substances which bind the GPR4 receptor, including inhibitors (e.g., antagonists) or promoters (e.g., agonists) of receptor function. In one embodiment, cells that naturally express GPR4 receptor protein or suitable host cells which have been engineered to express a GPR4 receptor or variant encoded by a nucleic acid introduced into those cells are used in an assay to identify and assess the efficacy of ligands, inhibitors or promoters of receptor function. Such cells are also useful in assessing the function of the expressed receptor protein or polypeptide.

Thus, the invention also relates to a method of detecting or identifying an agent that binds a mammalian GPCR such as GPR4 or ligand-binding variant thereof, comprising combining an agent to be tested, an antibody or antigen-binding fragment of the present invention and a composition comprising a mammalian GPR4 protein or a ligand binding variant thereof. The foregoing components can be combined under conditions suitable for binding of the antibody or antigen-binding fragment to mammalian GPR4 protein or a ligand binding variant thereof, and binding of the antibody or fragment to the mammalian GPR4 protein or ligand binding variant is detected or measured, either directly or indirectly, according to methods described herein or other suitable methods. A decrease in the amount of complex formed relative to a suitable control (e.g., in the absence of the agent to be tested) is indicative that the agent binds said receptor or variant. The composition comprising a mammalian GPR4 protein or a ligand binding variant thereof can be a membrane fraction of a cell bearing recombinant GPR4 protein or ligand binding variant thereof. The antibody or fragment thereof can be labeled with a label such as a radioisotope, spin label, antigen label, enzyme label, fluorescent group and chemiluminescent group. These and similar assays can be used to detect agents, including ligands (e.g., LPC or other inflammatory agents which interact with GPR4) or other substances, including inhibitors or promoters of receptor function, which can bind GPR4 and compete with the antibodies described herein for binding to the receptor.

The invention further relates to a method for determining whether a ligand is an agonist of the receptor according to the invention, which comprises preparing a cell extract from cells transfected with a vector expressing the nucleic acid molecule encoding a GPCR such as GPR4, isolating a membrane fraction from the cell extract, contacting the membrane fraction with the ligand under conditions permitting the activation of a functional receptor response and detecting by means of a bio-assay, such as a modification in the production of a second messenger an increase in the receptor activity, thereby determining whether the ligand is a receptor agonist.

The present invention additionally relates to a method for determining whether a ligand is an antagonist of the receptor according to the invention, which comprises contacting a cell transfected with a vector expressing the nucleic acid molecule encoding a GPCR such as GPR4 with the ligand in the presence of a known receptor agonist, under conditions permitting the activation of a functional receptor response and detecting by means of a bio-assay, such as a modification in second messenger concentration or a modification in the cellular metabolism, a decrease in the receptor activity, thereby determining whether the ligand is a receptor antagonist.

Preferably, the second messenger assay comprises measurement of intracellular cAMP, intracellular inositol phosphate (IP3), intracellular diacylglycerol (DAG) concentration or intracellular calcium mobilization. Other preferred second messenger molecules that can be assayed include the family of protein kinase C and Rho GTPases.

According to the present invention, ligands, inhibitors or promoters of receptor function can be identified in a suitable assay, and further assessed for therapeutic effect. Inhibitors of receptor function can be used to inhibit (reduce or prevent) receptor activity, and ligands and/or promoters can be used to induce (trigger or enhance) normal receptor function where indicated. These ligands, inhibitors and promoters can be used to treat inflammatory diseases, autoimmune diseases, atherosclerosis, and graft rejection, or HIV infection, for example, in a method comprising administering an inhibitor of receptor function to an individual (e.g., a mammal, such as a human). These ligands, inhibitors and promoters can also be used in a method of stimulating receptor function by administering a novel ligand or promoter to an individual, providing a new approach to selective stimulation of endothelial function, which is useful, for example, in the treatment of infectious diseases and cancer.

The present invention also encompasses a method of inhibiting leukocyte trafficking through blocking the activation of GPR4 expressed by vascular endothelium in a patient, comprising administering to the patient an effective amount of an antibody or functional fragment thereof that binds to a mammalian GPR4 or portion of said receptor and inhibits function associated with binding of a ligand to the receptor.

The present invention also relates to a method of inhibiting or treating GPR4-mediated disorders, such as inflammatory disorders, comprising administering to a patient an effective amount of an antibody or functional fragment thereof which binds to a mammalian GPR4 or portion of said receptor and inhibits GPR4-mediated function.

The present invention further relates to an antibody or fragment thereof as described herein for use in therapy (including prophylaxis) or diagnosis, and to the use of such an antibody or fragment for the manufacture of a medicament for the treatment of a GPR4-mediated disorder, or other disease or inflammatory condition as described herein.

### BRIEF DESCRIPTION OF THE DRAWINGS

In the drawings forming a portion of this disclosure,

FIG. 1 is a photograph of a gel that shows G protein-coupled receptor 4 (GPR4) mRNA expression in human cells. Cultured monolayers of human brain microvascular (HBMEC) or dermal (HMEC) endothelial cells were treated with 100 U/ml of tumor necrosis factor-α (TNF-α) or 50 micromolar/H₂O₂ for 2 hours or overnight (about 18 hours), and RT-PCR was performed from extracted total RNA. +Ctrl indicates positive control pAW109 RNA, 302 bp (GeneAmp RNA PCR kit); RT, murine leukemia virus reverse transcriptase; C, untreated control cell; lane 1=100 bp DNA ladder. Shown are bands at the predicted band size of 319 bp. Subsequent Basic Local Alignment Search Tool analysis of the sequenced bands indicated 96-97% identify with Human GPR4 gene database sequences and an E value <⁻¹⁶⁰. Negative controls (reaction performed in absence of RT) indicated absence of contaminating genomic DNA.

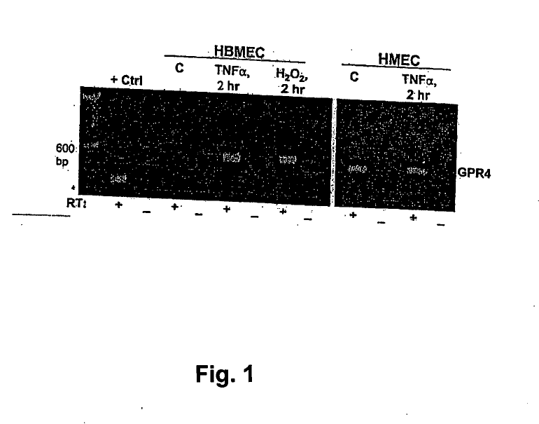

FIG. 2 in two parts shows detection of intact GPR4 protein with antibody to GPR4. COS7 cells were transfected with plasmid pEGFP-N1-3HA-GPR4 to over express GPR4. The cells were prepared for immunofluorescence evaluation (FIG. 2A) or Western blot analysis using affinity purified anti-GPR4 antibody referred to herein as anti-PepC (FIG. 2B). Immunofluorescence results in (FIG. 2A) demonstrated that transfected COS 7 cells activated green fluorescent protein (top panel); cells expressed GPR4 as detected by anti-PepC with red fluorescent protein (middle panel) and overlay of the images demonstrated that the transfected cells expressed GPR4 (bottom panel). Western blot analysis (FIG. 2B) demonstrated that only COS 7 cells transfected with pEGFP-N1-3HA-GPR4 had high expression of GPR4 as detected by anti-PepC whereas non-transfected and mock transfected (MK) cells showed barely detectable background signals.

FIG. 3 are photomicrographs that show immunofluorescent detection of endogenous GPR4 in endothelial cells. Confluent monolayers of human dermal microvascular endothelial cells were used to show that GPR4 is homogenously distributed in endothelial cells and dilution of anti-PepC (1:25 to 1:100) decreased the signal detected.

FIG. 4 in two parts shows Western blot analysis of endogenous GPR4 by endothelial cells. Again, human dermal microvascular endothelial cells were plated on a 6 well plate, grow to confluence then collected. Anti-PepC antibodies detected a band at about 45 KDa, the predicted size of GPR4 (FIG. 4A). Doubling the amount of protein lysate loaded on the gel showed a corresponding increase in the intensity of the band (FIG. 4A top panel). Blocking of anti-PepC with the GPR4 C terminal peptide eliminated the bands (FIG. 4A bottom panel). Anti-PepC antibodies cross-react with rat lung endothelial cells (RLEC), bovine pulmonary artery endothelial cells (BPAEC) and mouse kidney and lung tissues (FIG. 4B).

FIG. 5 are photomicrographs that show GRP4 expression in human brain tissue. Cryosections of human brain tissue (10-15 micrometers in thickness) were incubated with anti-PepC followed by a secondary anti-mouse antibody conjugated with rhodamine. Co-localization of the transmembrane glucose transporter, GLUT1 was also done by incubation with goat anti-GLUT1 antibody followed by a FITC labeled secondary antibody. GLUT1 is constitutively targeted to the plasma membrane of the vascular endothelium. Also DAPI is used for nuclei detection. The confocal image shows that GLUT1 (Left panel, FIG. 5A) and GPR4 are detected primarily within blood vessels (middle panel, FIG. 5B). An overlay (right panel, FIG. 5C) indicates that GPR4 is expressed primarily within vascular endothelium. The inset at the top (FIG. 5D) shows additional DAPI staining of nuclei in neuronal and glial cells.

FIG. 6 is a graph that shows expression of GPR4 mediates LPC-induced activation of the ICAM-1 promoter reporter gene. COS7 cells were transfected with full length ICAM-1 promoter, luciferase reporter gene construct, plus pEGFP-N1-3H-GPR4. The cells were stimulated with 15 micromolar LPC for 16 hours, luciferase activity determined and the relative light units (RLU) were normalized to protein. LPC stimulation significantly increased luciferase activity compared to either MK cells or control cells co-transfected with irrelevant DNA.

FIG. 7 shows Western blot analysis that illustrates siRNA induced knock down of GPR4 in endothelial cells. The retrovirus plasmid, pMSCVpuro-GPR4-siRNA and amphotropic packaging plasmid were co-transfected into 293T packaging cells to produce virus particles containing the small interference RNA targeted to GPR4 (siRNA-GPR4). The virus was harvested for infection of human dermal microvascular endothelial cells overnight (about 18 hours) to gene silence GPR4 and Western blot analysis with anti-PepC was performed. siRNA-GPR4 decreased by greater than 80% GPR4 expression in the endothelial cells compared to controls.

FIG. 8 is a graph that shows effects of siRNA-GPR4 on LPC-induced endothelial barrier dysfunction by examining the effects of siRNA-GPR4 on electrical transendothelial resistance over time. Endogenous GPR4 expressed in human dermal microvascular endothelial cells was knocked down as described above and the cells were stimulated with 15 micromolar LPC. Resistance was determined for about 3 hours. The LPC-induced resistance decrease was inhibited about 40-50% relative to control cells.

FIG. 9 is a graph that shows the effects of siRNA targeted to GPR4 on the in vitro monocyte transmigration across HBMEC after one and three hours. Retroviruses containing siRNA-GOR4 were used to infect HBMEC as described above. Control HBMEC show a normal basal level of monocyte transendothelial migration, which is increased with time. Pretreatment of HBMEC with LPC significantly increased monocyte transendothelial migration, particularly after 3 hours. HBMEC infected with siRNA-GPR4 did not alter the basal level of transmigration. HBMEC infected with siRNA-GPR4 resulted in prevention of the LPC induced increase in monocyte transmigration.

All amino acid residues identified herein are in the natural L-configuration. In keeping with standard polypeptide nomenclature, J. Biol. Chem., 243:3557-59, (1969), abbreviations for amino acid residues are as shown in the following Table of Correspondence:

TABLE OF CORRESPONDENCE
SYMBOL

| 1-Letter | 3-Letter | AMINO ACID |
|---|---|---|
| Y | Try | L-tyrosine |
| G | Gly | glycine |
| F | Phe | L-phenylalanine |
| M | Met | L-methionine |
| A | Ala | L-alanine |
| S | Ser | L-serine |
| I | Ile | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gln | L-glutamine |
| E | Glu | L-glutamic acid |
| W | Trp | L-tryptophan |
| R | Arg | L-arginine |
| D | Asp | L-aspartic acid |
| N | Asn | L-asparagine |
| C | Cys | L-cysteine |

### DETAILED DESCRIPTION OF THE INVENTION

Although this invention is susceptible of embodiment in many different forms, there is shown in the drawings and will herein be described in detail specific embodiments with the understanding that the present disclosure is to be considered as an exemplification of the principles of the invention and is not intended to limit the invention to the embodiments illustrated. It will also be understood that like or analogous elements and/or components, referred to herein, may be identified throughout the drawings with like reference characters.

G-protein coupled receptors (GPCRs) constitute a superfamily of integral membrane proteins with characteristic seven transmembrane domains. This superfamily includes receptors for a variety of biomolecules such as chemical messengers, bioactive amines, peptide hormones, neurotransmitters and even proteins [Eckard and Beck-Sickinger, Curr. Med. Chem. 7:897-910 (2000)].

One pharmaceutically important gene and protein for the treatment of asthma and disorders associated with defective cell signaling is the G protein-coupled receptor 4 (GPR4) gene and its encoded protein product. That encoded protein product contains a sequence 362 amino acid residues [Mahadevan et al., Genomics 30:84-88 (1995)]. GPR4 is a receptor with high mRNA expression levels seen in lung and to a lesser extent in kidney, heart, and selective brain regions. Sequence analysis suggests that GPR4 is a peptide receptor with 23-30 percent homology to receptors for purines, angiotensin II, platelet activating factor, thrombin, and bradykinin [Mahadevan et al., Genomics 30:84-88 (1995)]. The GPR4 gene is located in a region that is associated with susceptibility to asthma. Therefore, based on its chromosomal position and high expression in lung, GPR4 is likely to play a role in asthma.

A particularly preferred embodiment of the present invention contemplates a paratope-containing molecule such as an antibody that specifically binds to (immunoreacts with) human GPR4, and particularly binds to an epitope that is present in the carboxy-terminal (C-terminal) 50 residues of the molecule. More preferably, that epitope is present within the C-terminal approximately 40 amino acid residues of the sequence. Most preferably, that epitope is in the 11 residue sequence of positions 324 through 334 of the 362 residue sequence, and has the linear sequence, in single letter code, from left to right and in the direction from amino-terminus to carboxy-terminus,:

| | |
|---|---|
| ETPLTSKRNST | (SEQ ID NO: 1) |

In three-letter code, that sequence is

| | |
|---|---|
| GluThrProLeuThrSerLysArgAsnSerThr | (SEQ ID NO: 1) |

Interestingly, attempts to prepare anti-peptide antibodies that immunreact with the amino-terminus of the GPR4 sequence were unsuccessful using a peptide having the sequence of positions 2 through 9. Those two antibody preparations were referred to as being raised to otherwise unidentified peptides called as GPR4-A and GPR4-B, respectively in Qiao et al., Experimental Biology 2004: Meeting Abstracts 384.6 (2004). Antibodies that immunoreact with the peptide of SEQ ID NO:1 are sometimes referred to herein as anti-PepC.

As is illustrated hereinafter, contemplated antibodies, and particularly those raised to a polypeptide sequence of SEQ ID NO:1 are useful for assaying the presence and quantity of GPR4 in tissue samples. Thus, tissue samples from normal persons; i.e., those having no known disease state, and patients having a history of heart attack, stroke, arthritis, diabetes or the like diseases can be assayed to determine the relative amounts of GPR4 in tissues of persons free from known disease as compared to tissues of persons known to have had a particular disease, as well as tissues of persons recovering from a known disease.

Preparation of immunizing antigen (immunogen), and polyclonal and monoclonal antibody production can be performed as described herein, or using other suitable techniques. A variety of methods have been described (see e.g., Kohler et al, Nature, 256:495-497 (1975) and Eur. J. Immunol. 6:511-519 (1976); Milstein et al, Nature 266:550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al, Eds., (John Wiley & Sons: New York, N.Y.), Chapter 11, (1991)).

Generally for the production of a monoclonal antibody, a hybridoma can be produced by fusing a suitable immortal cell line (e.g., a myeloma cell line such as SP2/0) with antibody producing cells. The antibody producing cell, preferably from the spleen or lymph nodes, are obtained from animals immunized with the antigen of interest. The fused cells (hybridomas) can be isolated using selective culture conditions, and cloned by limiting dilution. Cells that produce antibodies with the desired binding properties can be selected by a suitable assay (e.g., ELISA). For a polyclonal antibody, a mammalian model such as a mouse, rabbit, sheep or pig is immunized with the antigen of interest. Serum is collected and polyclonal antibodies against the antigen are isolated using techniques well known in the art.

Other suitable methods of producing or isolating antibodies that bind GPR4, including human or artificial antibodies, can be used, including, for example, methods that select recombinant antibody (e.g., single chain Fv or Fab) from a library, or which rely upon immunization of transgenic animals (e.g., mice) capable of producing a repertoire of human or artificial antibodies (see e.g., Jakobovits et al., Proc Natl Acad Sci, USA, 90:2551-2555 (1993); Jakobovits et al., Nature, 362:255-258 (1993); Lonberg et al., U.S. Pat. No. 5,545,806; Surani et al., U.S. Pat. No. 5,545,807).

Single chain antibodies, and chimeric, humanized or primatized (CDR-grafted) antibodies, as well as chimeric or CDR-grafted single chain antibodies, and the like, comprising portions derived from different species, are also encompassed by the present invention. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Pat. No. 4,816,397; Boss et al, European Patent No. 0,120,694 B1; Neuberger, M. S. et al., WO 86/01533; Neuberger, M. S. et al, European Patent No. 0,194,276 B1; Winter, U.S. Pat. No. 5,225,539; Winter, European Patent No. 0,239,400 B1; and Queen et al., U.S. Pat. No. 5,585,089, No. 5,698,761 and No. 5,698,762. See also, Newman, R. et al., BioTechnology, 10:1455-1460 (1992), regarding primatized antibodies, and Ladner et al, U.S. Pat. No. 4,946,778 and Bird, R. E. et al., Science, 242:423-426 (1988)) regarding single chain antibodies.

In addition, functional fragments of antibodies, including fragments of chimeric, humanized, primatized or single chain antibodies, can be prepared. Functional fragments of the foregoing antibodies retain at least one binding function and/or modulation function of the full-length antibody from which they are derived. Preferred functional fragments retain an antigen-binding function of a corresponding full-length antibody (e.g., retain the ability to bind a mammalian GPR4). Particularly preferred functional fragments retain the ability to inhibit one or more functions characteristic of a mammalian GPR4, such as a binding activity, a signaling activity, and/or stimulation of a cellular response. For example, in one embodiment, a functional fragment can inhibit the interaction of GPR4 with one or more of its ligands, and/or can inhibit one or more receptor mediated functions of endothelial activation responses such as cytokine release, upregulation of adhesion molecules, upregulation of other pro-inflammatory genes, and impairment of barrier function.

Intact or whole antibodies that have their complete protein chains are contemplated herein, and are preferred. Antibody fragments capable of binding to a mammalian GPR4 receptor or portion thereof, including, but not limited to, Fv, Fab, Fab′ and F(ab′)₂ fragments are also encompassed by the invention. Such fragments can be produced by enzymatic cleavage or by recombinant techniques, for example. For instance, papain or pepsin cleavage can generate Fab or F(ab′)₂ fragments, respectively. Antibodies can also be produced in a variety of truncated forms using antibody genes in which one or more stop codons has been introduced upstream of the natural stop site. For example, a chimeric gene encoding a F(ab′)₂ heavy chain portion can be designed to include DNA sequences encoding the CH₁ domain and hinge region of the heavy chain.

Humanized immunoglobulins can be produced using synthetic and/or recombinant nucleic acids to prepare genes (e.g., cDNA) encoding the desired humanized chain. For example, nucleic acid (e.g., DNA) sequences coding for humanized variable regions can be constructed using PCR mutagenesis methods to alter DNA sequences encoding a human or humanized chain, such as a DNA template from a previously humanized variable region (see e.g., Kamman, M., et al., Nucl Acids Res, 17:5404 (1989)); Sato, K., et al., Cancer Research, 53:851-856 (1993); Daugherty, B. L. et al., Nucl Acids Res, 19:2471-2476 (1991); and Lewis, A. P. and J. S. Crowe, Gene, 101:297-302 (1991)). Using these or other suitable methods, variants can also be readily produced. In one embodiment, cloned variable regions can be mutagenized, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993; Knappik et al., WO 97/08320, published Mar. 6, 1997).

Anti-idiotypic antibodies are also provided. Anti-idiotypic antibodies recognize antigenic determinants associated with the antigen-binding site of another antibody. Anti-idiotypic antibodies can be prepared against second antibody by immunizing an animal of the same species, and preferably of the same strain, as the animal used to produce the second antibody. See e.g., U.S. Pat. No. 4,699,880.

Assays For GPR4

The polypeptides, antibodies and antibody combining sites (paratope-containing molecules) raised to the before described polypeptides, and methods of the present invention can also be used for diagnostic tests, such as immunoassays. Such diagnostic techniques include, for example, enzyme immune assay, enzyme multiplied immunoassay technique (EMIT), enzyme-linked immunosorbent (ELISA), radio-immune assay (RIA), fluorescence immune assay, either single or double antibody techniques, and other techniques in which either the paratope-containing molecule or the antigen is labeled with some detectable tag or indicating means. See generally Maggio, Enzyme Immunoassay, CRC Press, Cleveland, Ohio (1981); and Goldman, M., Fluorescent Antibody Methods, Academic Press, New York, N.Y. (1980). Specific examples of such assay methods and systems useful in carrying out those methods are discussed hereinbelow.

A method for assaying for the presence of GPR4 in a body sample is also contemplated herein. In a general method, a body sample to be assayed is provided, and is admixed with paratope-containing molecules to contact the sample with the paratope-containing molecules. The admixture is maintained for a predetermined period of time sufficient for the paratope-containing molecules to immunoreact with GPR4 present in the body sample. That maintenance time is typically about 5 to about 10 minutes to up to 24 hours and is typically at a temperature of about 4 degrees C. to about 45 degrees C. The amount of that immunoreaction is then measured to determine whether GPR4 molecules were present or absent in the assayed body sample, and in some cases the amount of GPR4 present in the sample.

An illustrative diagnostic system in kit form embodying one aspect the present invention that is useful for detecting GPR4 present in an aliquot of a body sample contains paratope-containing molecules of this invention such as antibodies, substantially whole antibodies, or antibody combining sites like Fab and F(ab′)₂ antibody portions. This system also includes an indicating means for signaling the presence of an immunoreaction between the paratope-containing molecule and the GPR4 antigen.

Typical indicating means include gamma-emitting radioisotopes such as ¹²⁴I, ¹²⁵I, ¹²⁸I and ¹³¹I, and ⁵¹Cr. Another group of useful labeling means are those elements such as ¹¹C, ¹⁸F, ¹⁵O and ¹³N themselves emit positrons. Also useful is a beta emitter, such as ¹¹¹In or ³H. Enzymes such as alkaline phosphatase, horseradish peroxidase, beta-D-galactosidase and glucose oxidase, and fluorochrome dyes such as fluorescein and rhodamine. The indicating means can be linked directly to paratope-containing molecule of this invention. The indicating means can also be linked directly to a separate molecule such as to a second antibody, to an antibody combining site or to Staphylococcus aureus (S. aureus) protein A that reacts with (binds to) the paratope-containing molecule of this invention. A specific example of such a separate molecule indicating means is ¹²⁵I-labeled S. aureus protein A.

The indicating means permits the immunoreaction product to be detected, and is packaged separately from the paratope-containing molecule when not linked directly to a paratope-containing molecule of this invention. When admixed with a body sample such as an acetone-fixed biopsied tissue sample, the paratope-containing molecule molecule immunoreacts with the GPR4 to form an immunoreactant, and the indicating means present then signals the formation of immunoreaction product.

One embodiment of a GPR4 diagnostic method is an immunofluorescent assay that includes an amplifying reagent. In such an assay a tissue sample is fixed to a plain microscope slide. An aliquot of antibodies raised in accordance with this invention generally about 10 micrograms to about 500 micrograms, is contacted with the slide using well-known techniques. After rinsing away any un-immunoreact antibodies of this invention, any non-specific binding sites on the slide are typically blocked with a protein such as bovine serum albumin (BSA) or powdered milk, if desired.

A second reagent (amplifying reagent) such as complement, or anti-immunoglobulin antibodies, e.g., guinea pig complement, can then be incubated on the test slide. After this second incubation, any unreacted amplifying reagent is removed as by rinsing leaving only that which is bound to the first-named antibodies on the assay slide. A third reagent (indicating means), e.g., antibody, like goat anti-guinea pig complement, is then incubated on the test slide. The third reagent is labeled by being linked to a fluorochrome dye such as fluorescein isothiocyanate (FITC), rhodamine β thiocyanate (RITC), tetramethylrhodamine isothiocyanate (TRITC), 4,4′-diisothiocyanostilbene-2,2′-disulfonic acid (DIDS), 5-dimethylamine-lnaphthalenesulfonyl chloride (DANSC), lissamine, rhodamine 8200 sulphonyl chloride (RB 200 SC) and the like as are well known in the art. A description of immunofluorescence analysis techniques is found in DeLuca, “Immunofluorescence Analysis”, in Antibody As A Tool, Marchalonis, et al., eds., John Wiley & Sons, Ltd., pp. 189231 (1982), which is incorporated herein by reference. Any unreacted third reagent is rinsed off after this third incubation, leaving any FITC labeled goat-antiguinea pig complement antibodies that bind to the complement on the test slide. The presence of the FITC labeled third reagent can be detected using flourescence microscopy and thereby signal the presence of EBV infection.

A preferred diagnostic system, preferably in kit form, useful for carrying out the above assay method includes, in separate packages, (a) paratope-containing molecules (e.g., antibodies) of this invention that immunoreact with GPR4, (b) a second, amplifying reagent such as complement, like guinea pig complement, anti-immunoglobulin antibodies or S. aureus protein A that reacts with the paratope-containing molecule, and (c) an indicating means that can be linked directly to the amplifying means or can be a portion of a separate molecule such as an antibody or antibody-portion that reacts with the amplifying reagent. The indicating means indirectly signals the immunoreaction of the paratope-containing molecule and GPR4 through the mediation of the amplifying reagent.

Paratope-containing molecule molecules and separate indicating means of any diagnostic system described herein, as well as the above-described amplifying reagent, can be provided in solution, as a liquid dispersion or as a substantially dry powder, e.g., in lyophilized form. Where the indicating means is a separate molecule from the amplifying reagent, it is preferred that the indicating means be packaged separately. Where the indicating means is an enzyme, the enzyme's substrate can also be provided in a separate package of the system. A solid support such as the before-described microscope slide, one or more buffers and acetone can also be included as separately packaged elements in this diagnostic assay system.

The packages discussed herein in relation to diagnostic systems are those customarily utilized in diagnostic systems. Such packages include glass and plastic (e.g., polyethylene, polypropylene, polystyrene and polycarbonate) bottles, vials, plastic and plastic-foil laminated envelopes and the like.

The use of whole, intact, biologically active antibodies is not necessary in many diagnostic systems such as the immunoflourescent assay described above. Rather, only the immunologically active, paratope-containing molecule site; i.e., the antibody combining site, of the antibody molecule can be used. Examples of such antibody combining sites are those known in the art and are discussed elsewhere herein

A wide variety of molecules can be assayed for their ability to modulate the immune system. Representative examples that are discussed in more detail below include organic molecules, proteins or peptides, and nucleic acid molecules. U.S. Pat. No. 6,770,449 describes numerous assays that are available for screening compound binding to GPCR in cells.

Numerous organic molecules can be assayed for their ability to modulate the immune system. For example, within one embodiment of the invention suitable organic molecules can be selected either from a chemical library, wherein chemicals are assayed individually, or from combinatorial chemical libraries where multiple compounds are assayed at once, then deconvoluted to determine and isolate the most active compounds.

Representative examples of such combinatorial chemical libraries include those described by Agrafiotis et al., U.S. Pat. No. 5,463,564; Armstrong, WO 95/02566; Baldwin et al., WO 95/24186; Baldwin et al., WO 95/30642; Brenner, WO 95/16918; Chenera et al., WO 95/16712; Ellman, U.S. Pat. No. 5,288,514; Felder et al., WO 95/16209: Lerner et al., WO 93/20242; Pavia et al., WO 95/04277; Summerton et al., U.S. Pat. No. 5,506,337; Holmes, WO 96/00148; Phillips et al., Tet. Letters 37:4887-4890, 1996; Ruhland et al., J Amer Chem Soc 111:253-254, 1996; Look et al., Bioorg and Med Chem Letters 6:707-712, 1996.

Similarly, a wide range of proteins and peptides can be utilized as candidate molecules for modulating the immune system. Peptide molecules that modulate the immune system can be obtained through the screening of combinatorial peptide libraries. Such libraries can either be prepared by one of skill in the art (see e.g., U.S. Pat. No. 4,528,266 and No. 4,359,535, and Patent Cooperation Treaty Publications WO 92/15679, WO 92/15677, WO 90/07862, WO 90/02809, or can be purchased from commercially available sources (e.g., New England Biolabs Ph.D.™ Phage Display Peptide Library Kit).

Several studies have implicated RhoA activation in endothelial cells in the regulation of leukocyte transmigration [Adamson et al., J Immunol 162:2964-2973 (1999); Strey et al., FEBS Lett 517:261-266 (2000)]. Further, previous work from the inventor's laboratory [Huang et al., Am J Physiol 289:L176-L185 (2005)] indicates that LPC activates RhoA in endothelial cells, a signaling pathway known to be critical in the regulation of vascular endothelial barrier dysfunction. Whether the LPC-stimulated monocyte transmigration is dependent on the RhoA signaling cascade in HBMEC was investigated.

HBMEC were pretreated with 5 mg/ml for 24 hours of C3 transferase toxin (Clostridium botulinum) (Biomol, Plymonth Meeting, Pa.) to inactivate RhoA, B, and C by ADP ribosylation of Rho at asparagine 41 [Lerm et al., FEMS Microbiol Lett 188:1-6 (2000)]. The C3 concentration used is a maximum concentration determined for endothelial cells in the previous study [Huang et al., Am J Physiol 289:L176-L185 (2005)].

Results reveal that the C3 transferase pretreatment inhibited about 65% of the LPC-induced monocyte transmigration. C3 transferase alone did not change basal monocyte transmigration. These findings indicate that RhoA mediated, at least in part, the LPC-induced monocyte transmigration across HBMEC.

To test whether the LPC-induced monocyte transmigration is dependent on GPR4, a recombinant retrovirus containing siRNA targeted to GPR4 (siRNA-GPR4) was used to post-transcriptionally induce gene silencing of endogenous GPR4 in HBMEC. This approach has been successfully used to knock down GPR4 expression in human dermal microvascular endothelial cells [Kim et al., FASEB J. 19:819-821(2005)]. Infection of endothelial cells with the retrovirus was well-tolerated and cells did not show significant changes of morphology. In HBMEC, infection with siRNA-GPR4 resulted in about a 62% decrease in GPR4 protein expression compared to either non-infected control cells or HBMEC infected with siRNA-LPA3, which was targeted to a different G protein-coupled receptor, LPA3 as determined by Western blot using the before-described anti-GPR4 antibodies. The results indicated that siRNA-GPR4 could effectively reduce GPR4 expression in HBMEC and was specific for GPR4.

To determine the effects of GPR4 knock down on the LPC-induced transmigration response, HBMEC were treated with siRNA-GPR4 to induce knock down of GPR4, then the HBMEC were stimulated with LPC, and monocyte transmigration determined as described. The results indicated that decreased GPR4 expression corresponded with about 90% decreased LPC-stimulated monocyte transendothelial migration. As negative control, parallel studies with infection with siRNA-LPA3 of HBMEC resulted in similar LPC-stimulated increases in monocyte transmigration as across control non-infected HBMEC. These latter findings suggest that retrovirus infection of HBMEC per se did not inhibit monocyte transmigration, rather the selective knock down of GPR4 prevented the transmigration.

For study, HBMEC were infected with siRNA-GPR4 (as described for the transmigration assay), then stimulated with LPC, and RhoA affinity binding assay was made to determine RhoA activation. Results showed that LPC caused a significant increase in RhoA-GTP in HBMEC. Following knock down of GPR4 with siRNA, the LPC-stimulated increase in RhoA-GTP was effectively abrogated. As negative control, HBMEC were infected with the siRNA-LPA3, and subsequent results indicated absence of inhibition of the LPC-induced RhoA-GTP increase. These studies provide evidence that LPC-mediated RhoA activation occurs through GPR4 expression in HBMEC in the regulation of monocyte transmigration.

### EXAMPLE 1

### Antibody Preparation

Polyclonal antibodies to GPR4 were made at the Research Resources Center, University of Illinois at Chicago. Peptides corresponding to either N-terminus (positions 2 through 9) or C-termini (positions 324 through 334) of human GPR4 (GenBank Number U21051) were synthesized on an Applied Biosystems Peptide Synthesizer (Model 433; Foster City, Calif.) using solid phase peptide synthesis with Fmoc (9-fluorenylmethl-oxycarbonyl) chemistry. The peptide was checked and verified by its single peak in the analytical HPLC chromatogram, amino acid composition, and mass spectrum and by NH₂-terminal sequencing.

Each peptide was separately conjugated to keyhole limpet hemocyanin (KLH) using the heterobifunctional coupling reagent m-maleimidobenzoyl-N-hydroxysuccinimide ester (MBS) dissolved (5 mg) in 0.5 ml 0.01 mol/L phosphate buffer (pH 7.0) for immunization in rabbits. Blood was collected before injection to obtain preimmune serum from the rabbits. Booster injections were given at 4 week intervals, and blood collected 3-4 weeks after each immunization.

The anti-peptide sera can be purified using routine peptide affinity column chromatography. In brief, the immunizing peptide is coupled to Sepharose 4B gel in ligand coupling buffer (0.1 M NaHCO₃, pH 8.3, containing 0.5 ml NaCl), loaded into 10 cm column, and washed with 100×bed volume of PBS. The filtered antiserum is loaded into column, washed, and antibodies are eluted with glycine buffer, pH2.5 (50 mM glycine-HCl, pH 2.5; 0.1% Triton X100; 0.15 M NaCl). The collected antibodies are desalted in PD-10 columns.

Immunoreactivity of the antiserum to the GPR4 peptide antigens was evaluated by indirect ELISA. Microtiter plates (96-well) were coated with the GPR4 C-terminal peptide or GPR4 N-terminal peptide per well. Two-fold serial dilutions of anti-GPR4 peptide antiserum or dilution of preimmune serum were added to appropriate wells, followed by incubation of goat anti-rabbit IgG conjugated to alkaline phosphatase. The enzyme substrate, p-nitrophenyl phosphate, was added to each well. The reaction was detected by reading absorbency at 405 nm.

Results indicated that the anti-GPR4 serum prepared from the C-terminal sequence (positions 324 through 334) was antigenic and detected the C-terminal GPR4 peptide compared with preimmune serum. The effective dilution was 1:100 and higher dilutions (>1:1,000) showed minimal antigenicity. Comparable dilutions of anti-GPR4 antisera prepared with the N-terminal peptide lacked antigenicity. Therefore, the anti-C-terminus peptide antiserum was affinity-purified by peptide affinity column chromatography for use herein.

### EXAMPLE 2

### GPR4 Expression in HBMEC

Human microvascular endothelial cells from brain (HBMEC) were cultured to elucidate the induced expression of GPR4 in HBMEC with inflammatory mediators such as TNF-α and oxidants. HBMEC were grown in RPMI 1640 supplemented with 10% FBS, 10% NuSerum (Becton Dickinson; Bedford, Mass.), endothelial cell growth supplement (30 μg/ml), heparin (5 U/ml), 1 mmol/l sodium pyruvate, 1 mmol/l minimal essential media (MEM), nonessential amino acids, 1 mmol/l MEM vitamins, 1% L-glutamine, and 1% penicillin-streptomycin.

The expression of GPR4 mRNAs in HBMEC was determined by RT-PCR [Lum H, et al., Am J Physiol Cell Physiol 282:C59-C66 (2002)]. HBMEC were treated with 2 hours or overnight (about 18 hours) with either TNF-α (100 U/ml) or H₂O₂ (50 pmol/l), and total RNA extracted as well from human lymphocytes as a positive control. Total RNA was reverse transcribed with oligo-dT primers, and PCR was performed with specific primer sets corresponding to GenBank sequences of human GPR4. Consequently, RT-PCR products were analyzed by 1.5% agarose gel electrophoresis.

### EXAMPLE 3

### Specific Binding of LPC to Endothelial Cell

### Surface by Competition Binding Assay

Confluent cell monolayers grown in 24-well culture dishes were treated overnight (about 18 hours) with either TNF-α (100 U/ml) or H₂O₂ (50 μmol/l). The cells were washed and incubated for 60 minutes at 4° C. with HEPES buffer (pH 7.4, 0.1% BSA) containing 0.02 nmol [³]H-LPC plus a 200-fold molar excess of unlabeled LPC. After three washes with cold HEPES buffer, cells were lysed with 0.1 mol/l NaOH, radioactivity was counted, and specific binding from duplicate samples was calculated as (fmol LPC bound/10⁶ cells). Separate dishes of cells were treated in parallel for cell count determination.

### EXAMPLE 4

### LPC Receptor GPR4 mRNA Expression in Brain and Skin

Human endothelial cells from brain (HBMEC) and skin (HMEC) expressed the LPC receptor GPR4. This selectivity for GPR4 expression by endothelial cells is consistent with the report that GPR4 appears to have wide tissue distribution, including the ovary, lung, kidney, liver, brain, and lymph nodes (Zhu K, et al, J Biol Chem 276:41325-41335, 2001). This wide distribution of GPR4 receptors extends to the vascular endothelium. It has been further determined that inflammatory stress induces GPR4 expression in HBMEC and HMEC. It is known to those having ordinary skill in the art that LPC receptors can be induced by a wide range of signals, including but not limited to, DNA-damaging reagents, stress, and apoptosis (Weng Z, et al, Proc Natl Acad Sci, USA 95:12334-12339, 1998).

HBMEC and HMEC were stimulated by the cytokine TNF-α or H₂O₂ for 2 hours or overnight (about 18 hours). The results indicated that in HBMEC, but not HMEC, stimulation with TNF-α or H₂O₂ increased GPR4 mRNA over control within 2 hours. Subsequent sequencing of the purified bands in both forward and reverse directions (Research Resources Center, University of Illinois at Chicago) and BLAST 2.0 analysis (Basic Local Alignment Search Tool, NCBI) indicated that the GPR4 DNA sequences had 96-97% identity with gene database sequences, corresponding to E values <10⁻¹⁶⁰. This result shows that GPR4 was increased by TNF-α or H₂O₂ stimuli. Therefore, the results suggest that cerebral vascular endothelium appear to be highly sensitive to inflammatory stresses in the context of LPC receptor expression, which can lead to enhanced responsiveness to LPC.

### EXAMPLE 5

### Antibody Detection of GPR4

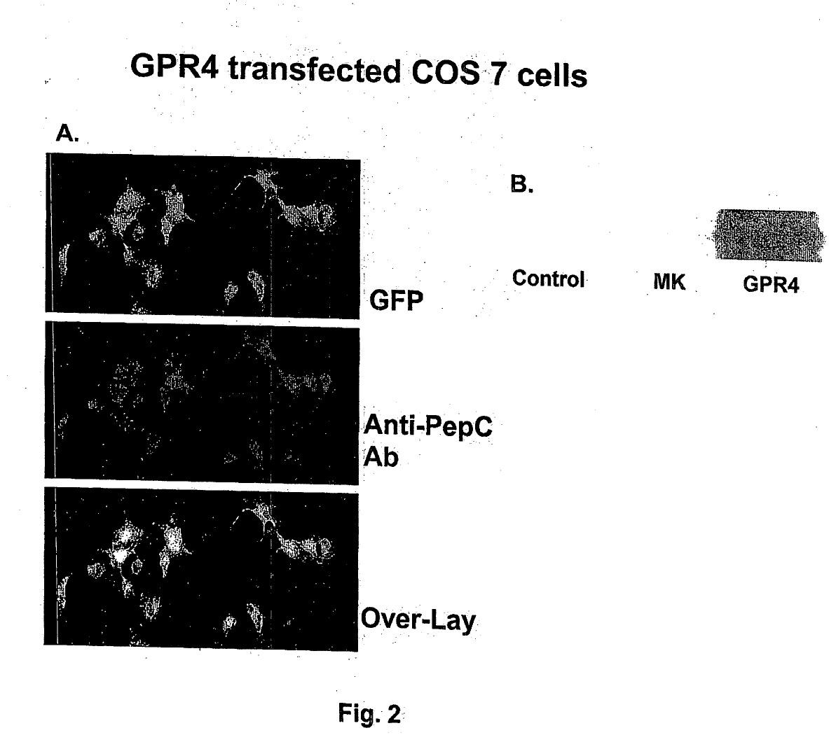

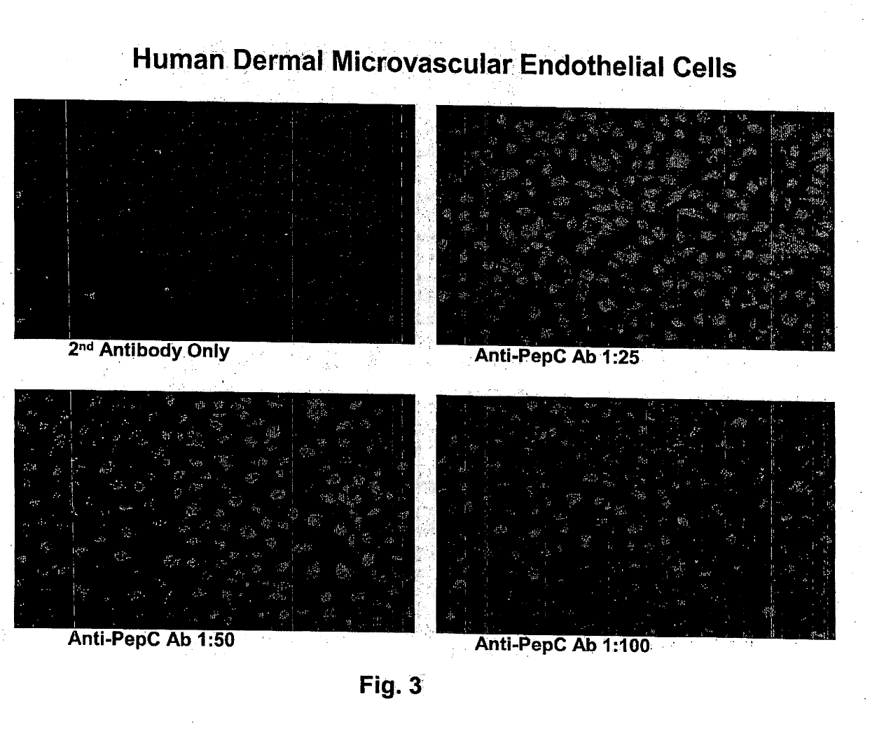

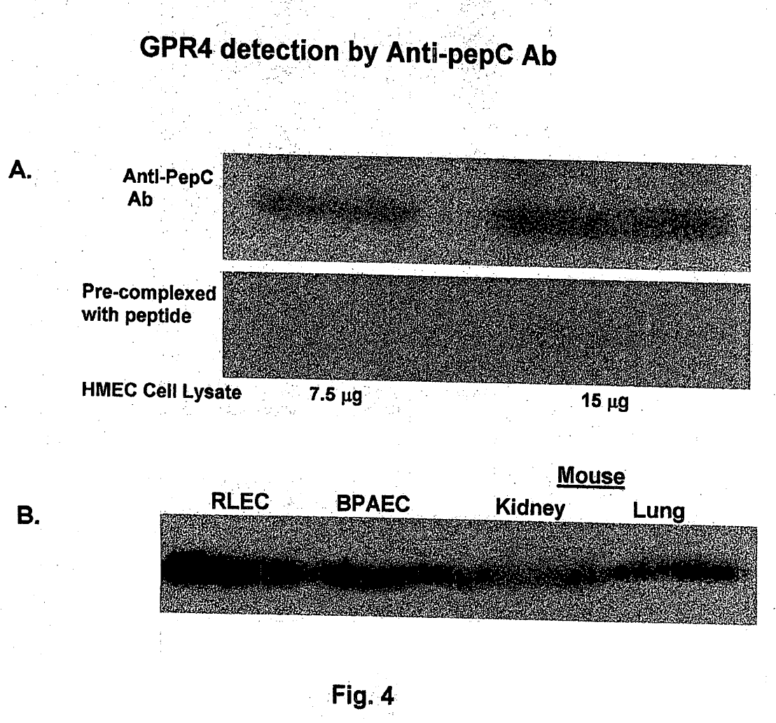

An exemplary antibody prepared as above to the GPR4 C-terminal peptide was successfully able to detect intact GPR4 in COS 7 cells transfected with a plasmid containing the GPR4 RNA (FIG. 2) through immunofluorescence and Western blotting. This result demonstrates the specificity of the antibody and serves as a control for studies in target cells. The C-terminal antibody (anti-PepC) was then shown to detect endogenous GPR4 in endothelial cells using the same detection techniques (FIGS. 3 and 4). This result demonstrates that GPR4 is found on cultured vascular endothelial cells.

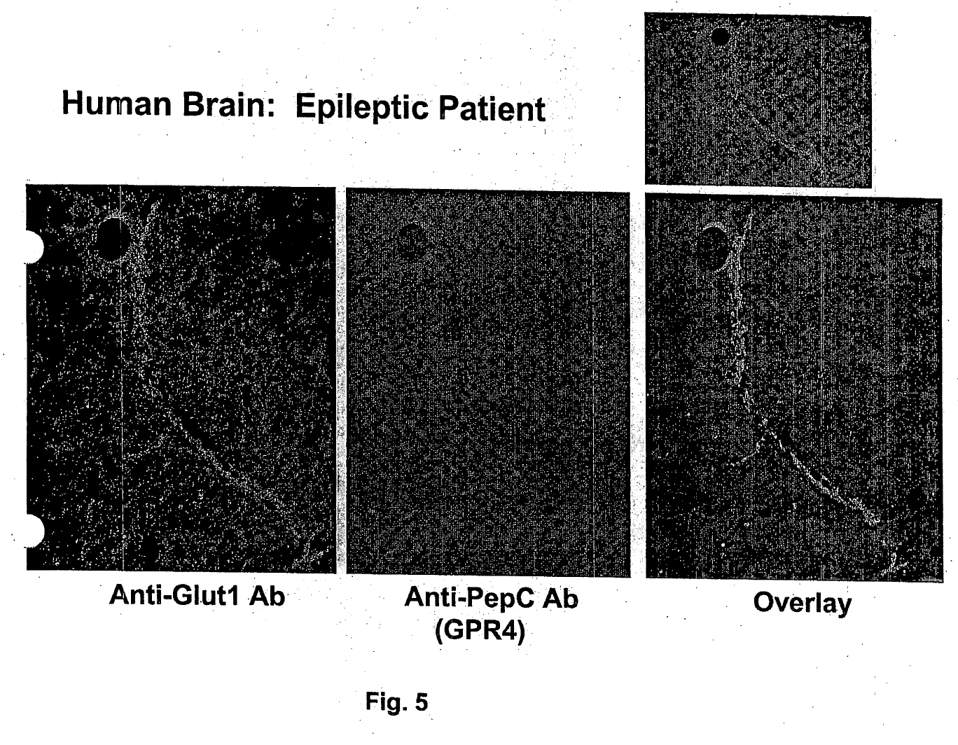

The anti-GPR4 antibody (anti-PepC) was then assayed in cryosections of human brain tissue (FIG. 5). Immunohistochemistry studies of the sections demonstrate that the receptor localizes within blood vessels and is expressed primarily within the vascular endothelium in tissue.

### EXAMPLE 6

### Regulation of GPR4 Expression

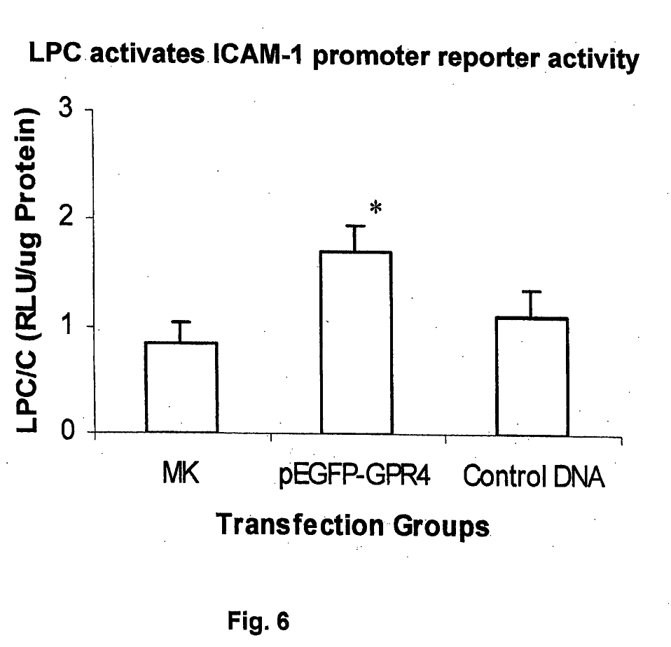

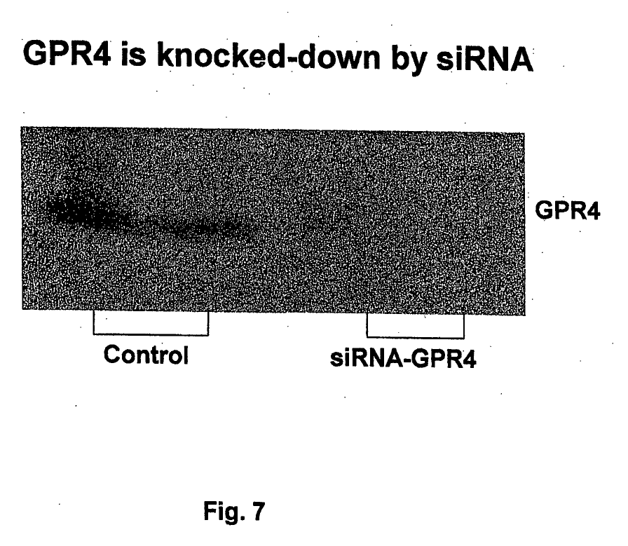

Studies using a luciferase reporter gene (FIG. 6) and siRNA knockdown of GPR4 (FIG. 7) demonstrated that the GPR4 receptor could be manipulated chemically by using LPC (up-regulated) or small interference RNA targeted to GPR4 (down-regulated). Thus several assays are provided to demonstrate the effect of target molecules on the regulation and expression of GPR4.

### EXAMPLE 7

### Endothelial Barrier Dysfunction

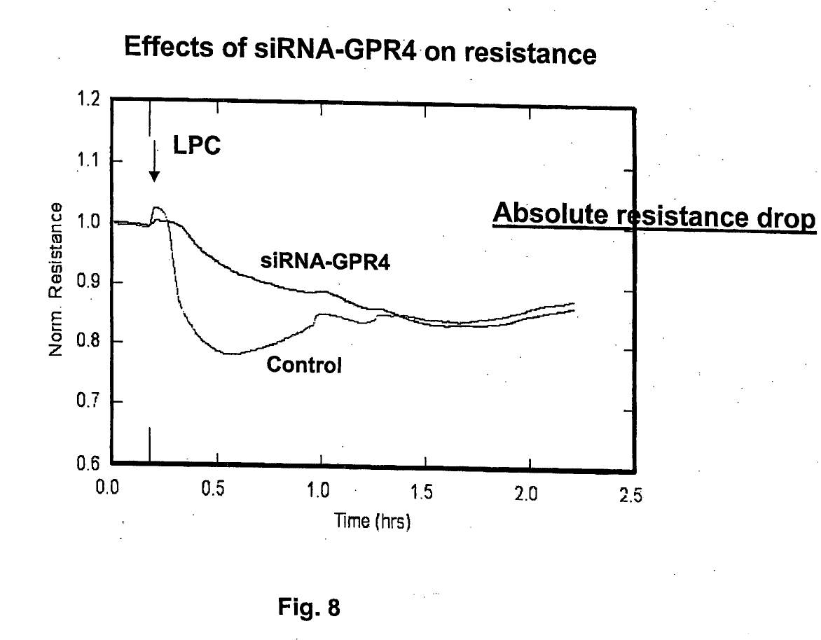

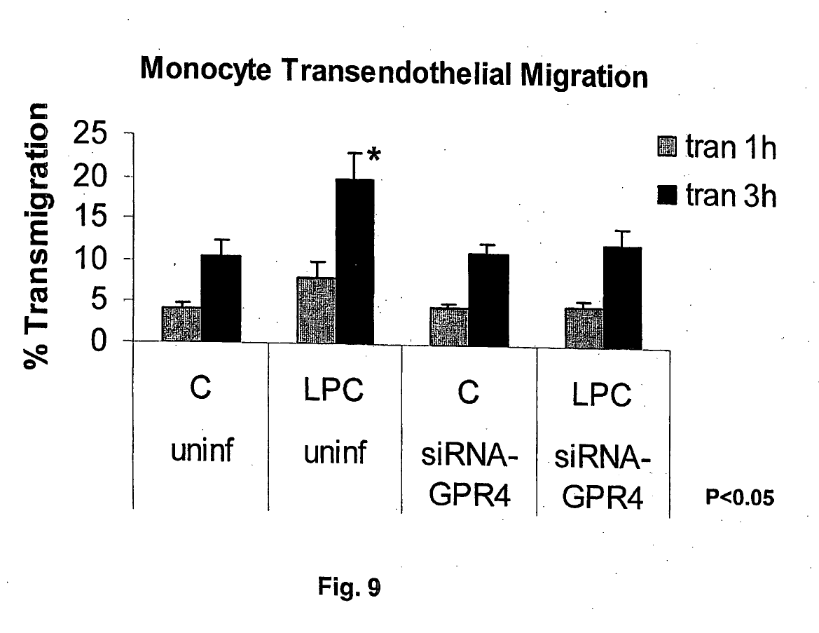

LPC-induced endothelial barrier dysfunction studies and monocyte transmigration studies (FIGS. 8 and 9) were performed to demonstrate the physiological effect of inhibitors (siRNA-GPR4) and activators (LPC) of GPR4 alone or in combination. Again, these techniques provide an in vitro method for monitoring the physiological effect of activators or inhibitors of GPR4 in the presence or absence of LPC. These methods are useful for screening compounds for use as anti-inflammatory agents.

### EXAMPLE 8

### Monocyte Migration Studies Preparation of LPC

LPC (1-palmitoyl-2-hydroxy-sn-glycero-3-phosphocholine) was purchased from Avanti Polar Lipids, Alabaster, Ala., checked for fatty acid composition by gas liquid chromatography, and found to be at least 96% pure. It was dissolved in chloroform:methanol (2:1) and stored at −20° C. Aliquots were evaporated under nitrogen in glass tubes, and resuspended in sufficient volume of Hanks Balanced Salt Solution (HBSS) to give a final concentration of 1 mM. The samples were vortexed at room temperature for 1 minute (2×) to yield a clear dispersion and the final concentration was confirmed by analysis of lipid phosphorus by the modified Bartlett procedure [Marinetti, J Lipid Res; 3:1-20(1962)]. The phospholipid dispersions were stored at 4° C., and were used within 30 days of the preparation.

Cell Culture

Human brain microvascular endothelial cells (HBMEC) were grown in RPMI 1640 supplemented with 10% fetal bovine serum (FBS), 10% NuSerum (Becton Dickinson, Bedford, Mass.), endothelial cell growth supplement (30 μg/ml), heparin (5 U/ml), 1 mM sodium pyruvate, 1 mM MEM non-essential amino acids, 1 mM MEM Vitamins, 1% L-glutamine, and 1% penicillin-streptomycin. Cells were cultured at 37° C. in a humidified CO₂ incubator at 5% CO₂. The cultured HBMEC express both endothelial cell phenotypic and functional characteristics [Stins et al., [letter] In Vitro Cellular & Developmental Biology; Animal. 33:243-247 (1997); Stins, J Neuroimmunol; 76:81-90 (1997)]. COS7 cells and 293T cells were maintained in Dulbecco's Modified Eagle's Medium containing 4.5 g/liter glucose, 5% FBS, and 1% penicillin-streptomycin.

Isolation and Labeling of Monocytes

Human monocytes were freshly isolated from whole blood obtained from healthy donors. Blood was added to the RosetteSep™ human monocyte enrichment cocktail (StemCell Technologies Inc., Canada) and incubated for 20 minutes at room temperature. Then the sample was diluted with an equal volume of PBS containing 2% FBS and 1 mM EDTA, layered on top of Ficoll-Paque™ Plus, and the cell suspension was centrifuged at 1200×g for 20 minutes. The enriched cells were collected from the Ficoll-Paque/plasma interface, and lysed with ammonium chloride to remove residual red blood cells. The monocytes were labeled with fluorescent dye calcein (Molecular Probes, Eugene, Oreg.) as follows: DMSO (5 μl), 20% plurionic acid (5 μl), heat-inactivated FBS (60 μl), and 50 μg of calcein. The monocytes were incubated at room temperature in a rotor plate for 50 minutes, then centrifuged and supernatant removed. The cells were washed twice with Ca²⁺-free HBSS, and resuspended in HBSS containing 1% FBS for studies.

Monocyte Transmigration Assay

HBMEC were plated at 0.8×10⁵ cells/transwell filter (6.5 mm diameter, 5.0 μm pore size; Corning Costar Corporation, Cambridge, Mass.) and grown to confluence on fibronectin-coated transwells. Transwell filters were suspended in 24-well culture plates so that the filter separated the upper and lower compartments. The HBMEC were challenged with 5 μM LPC at 37° C. for the indicated times, after which they were washed with HBSS to remove LPC. The calcein-labeled human monocytes were added onto the HBMEC monolayer in fresh medium at 2×10⁵ cells/well and incubated for up to 3 hours at 37° C. to permit transmigration into lower well. After incubation, total fluorescence (from 2×10⁵ monocytes) and the fluorescence of the monocytes transmigrated to the bottom well were measured in a Cytofluor plate reader (PreSeptive Biosystems, Framingham, Mass.). The percentage of monocyte transmigration was calculated by the fluorescence of the transmigrated monocytes divided by total fluorescence times 100. The studies were made in triplicates.

Affinity-Binding Assay for RhoA Activation

The GTP-bound form of RhoA was determined by affinity-binding assay to evaluate RhoA activation as previously described [Huang et al., Am J Physiol; 289:L176-L185 (2005); Qiao et al., Am J Physiol; 284:L972-L980 (2003)]. The assay is based on the use of the plasmid pGST-C21 (glutathione-S-transferase-C21 fusion protein, generously provided by Dr. John G. Collard, The Netherlands Cancer Institute, Amsterdam), which contains a 291-base pair insert from rhotekin, a Rho target molecule, that binds strongly to activated RhoA.

DH5a competent E. coli cells were transformed with pGST-C21. Bacterial-expressed GST-rhotekin was induced by addition of 0.1 mM isopropylthiogalactoside. HBMEC were grown in 6-well dishes to confluence, treated according to experimental protocol, and collected in GST-FISH buffer [50 mM Tris (pH 7.4), 10% glycerol, 100 mM NaCl, 1% Nonidet NP-40, 2 mM MgCl₂, 25 mM NaF and 1 mM EDTA] plus protease inhibitor cocktail (10 μg/ml of pepstatin A, 10 μg/ml each of aprotinin and leupeptin, and 1 mM PMSF). Cell lysates were pelleted by centrifugation at 10,000 g at 4° C. for 5 minutes, and equal volumes of supernatant were incubated with purified GST-rhotekin coupled to glutathione Sepharose™ 4B beads (Amersham Pharmacia Biotech, Piscataway, N.J.) at 4° C. for 1 hour. The GTP-form of RhoA bound specifically to the rhotekin-Sepharose beads was eluted by boiling in 2.5× Laemmli sample buffer. The eluted sample and total cell lysate were electrophoresed on 12.5% SDS-PAGE, and Western blot analysis made with affinity-purified antibody directed against RhoA (Santa Cruz Biotechnology, San Diego, Calif.). The GTP-bound RhoA was quantified by scanning densitometry.

Production of siRNA Retrovirus

The retrovirus plasmid (pMSCVpuro-GPR4—RNAi) contains the small interference RNA (siRNA) targeted to GPR4 [Kim et al., FASEB J. 19:819-821(2005)] and was used to gene silence endogenous GPR4 expression in HBMEC. The pMSCVpuro-GPR4—RNAi and the amphotropic packaging plasmid (each 1 μg/ml plasmid) were co-transfected using Lipofectamine (5 μl/ml) into 293 T packaging cells at 70-80% confluence with serum-free DMEM. After 3 hours of incubation at 37° C., fresh complete medium (DMEM containing 10% FBS) was added. Twenty-four hours later, the medium was changed to fresh complete DMEM, and incubated overnight for virus production. The virus particles (siRNA-GPR4) were harvested from medium, filtered, and used for infection of HBMEC. For negative control, pMSCV-LPA3-RNAi, which contains siRNA targeted to LPA3 receptor, was similarly used to generate recombinant retrovirus containing siRNA-LPA3. These retrovirus plasmids were generously provided by Dr. Yan Xu, The Cleveland Clinic Foundation, OH.

Western Blot

HBMEC were grown to confluence and treated according to experimental protocol. The cells were washed twice with PBS and collected in the appropriate extraction buffer and protein concentration determined using the BCA Protein Assay kit with bovine serum albumin as standard (Pierce, Rockford, Ill.). The cell lysates were loaded at constant protein concentrations, separated by SDS-polyacrylamide gel electrophoresis containing 12% acrylamide, and electrotransferred to nitrocellulose membrane. The membrane was blocked with 5% nonfat dry milk in Tris buffered saline with 0.05% Tween-20 (TBST), then incubated with affinity-purified primary antibodies diluted in TBST with 1% nonfat dry milk for overnight at 4° C. in a rocker. The blot was washed 5× with TBST and incubated with the appropriate anti-IgG secondary antibody conjugated with horseradish peroxidase. To evaluate equal loading of proteins per lane, membranes were stripped and reprobed for β-actin with monoclonal anti-β-actin antibody (Sigma, St. Louis, Mo.). The bands were detected using the enhanced chemiluminescence kit (ECL from Amersham).

Reporter Assay

COS7 cells were plated in 6-well dishes and grown to 70-80% confluence. The cells were co-transfected with 2 μg of pEGFP-N1-3HA-GPR4 plus 2 μg of the ICAM-1 luciferase (ICAM-1 LUC) reporter plasmid using 7.5 μl Lipofectamine™ reagent. The ICAM-1 LUC reporter plasmid contains the full-length ICAM-1 promoter linked to the firefly luciferase as previously described [Roebuck et al., J Biol Chem; 270:18966-18974 (1995)]. Control transfectants were co-transfected with equal amounts of a non-relevant plasmid (pRL-TK) as a control for the amount of transfected DNA. Mock transfected COS7 cells were treated with Lipofectamine only without DNA. After 3 hours of incubation at 37° C., the medium was replaced with DMEM containing 10% FBS. After incubation overnight (about 18 hours), the cells were checked under fluorescent microscopy to determine the expression of GPR4 by examining for green fluorescent protein. The cells were then stimulated with LPC in DMEM containing 10% FBS and collected for assay of luciferase activity using the Luciferase Assay Kit (Promega, Madison, Wis.) according the manufacture's protocol. The luciferase activity was measured with a Femtomaster FB12 luminometer (Zylux Corporation, Maryville, Tenn.). The transfection efficiency did not varied significantly between experiments and therefore, luciferase activity is reported as relative light units (RLU) normalized to protein.

Statistics

Single sample data were analyzed by the two-tail t test; a multiple range test (Scheffe's test) was used for comparisons of experimental groups with a single control group.

Each of the patents and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

What is claimed:

1. A paratope-containing molecule that specifically binds to human GPR4.

2. The paratope-containing molecule according to claim 1 that is an antibody.

3. The paratope-containing molecule according to claim 1 that specifically binds to an epitope present in the C-terminal 50 residues of human GPR4.

4. The paratope-containing molecule according to claim 3 that specifically binds to an epitope present in the C-terminal 40 residues of human GPR4.

5. A paratope-containing molecule that specifically binds to an epitope present in the C-terminal 40 residues of human GPR4.

6. The paratope-containing molecule according to claim 5 that specifically binds to an epitope present in a peptide having the sequence, from left to right and in the direction from amino-terminus to carboxy-terminus, of GluThrProLeuThrSerLysArgAsnSerThr (SEQ ID NO:1).

7. The paratope-containing molecule according to claim 5 that is a Fv, Fab, Fab′ or F(ab′)₂ antibody fragment.

8. The paratope-containing molecule according to claim 5 that is an intact antibody.

9. A method for assaying for the presence of human GPR4 in a body sample that comprises the steps of:
(a) contacting a body sample to be assayed with paratope-containing molecules that specifically bind to human GPR4;
(b) maintaining that contact for a time period sufficient for human GPR4 present in the sample to specifically bind to paratope-containing molecules to form an immunocomplex; and
(c) determining the presence of a formed immunocomplex and thereby the presence of GPR4.

10. The method according to claim 9 wherein said body sample is a tissue sample.

11. The method according to claim 9 wherein the presence of a formed immunocomplex is signaled by an indicating means.

12. The method according to claim 11 wherein the indicating means is linked directly to paratope-containing molecule.

13. The method according to claim 11 wherein the indicating means is linked to a separate molecule.

14. The method according to claim 9 wherein said paratope-containing molecules specifically bind to an epitope present in the C-terminal 50 residues of human GPR4.

15. The method according to claim 14 wherein said paratope-containing molecules specifically bind to an epitope present in a peptide having the sequence, from left to right and in the direction from amino-terminus to carboxy-terminus, of GluThrProLeuThrSerLysArgAsnSerThr (SEQ ID NO:1).

16. A diagnostic system in kit form useful for assaying for the presence of GPR4 that includes, in a package,
(a) paratope-containing molecules that immunoreact with GPR4 and utilize an indicating means that signals the presence of an immunocomplex formed between GPR4 and the paratope-containing molecules.

17. The kit according to claim 16 that further includes (b) a second, amplifying reagent that reacts with the paratope-containing molecule and is present in a separate package.

18. The kit according to claim 17 wherein said indicating means that signals the presence of an immunocomplex formed between GPR4 and the paratope-containing molecules acts through the mediation of the amplifying reagent.

19. The kit according to claim 18 wherein said indicating means is linked directly to said amplifying agent.
